(19) United States
(12) Patent Application Publication
(10) Pub. No.: US 2006/0189857 A1
(43) Pub. Date: Aug. 24, 2006

(54) System and method for monitoring or treating a health condition

(75) Inventors: Mark Faupel, Alpharetta, GA (US); Raulee Marcus, Hermosa Beach, CA (US); Alan Smith, Atlanta, GA (US); Difei Yang, Alpharetta, GA (US)

Correspondence Address: Michael B. Lasky;Altera Law Group, Suite 100, 6500 City West Parkway, Minneapolis,, MN, 55344-7704, US

(73) Assignee: SpectRx, Inc.

(21) Appl. No.: 11/412,491

(22) Filed: Apr. 27, 2006

Related U.S. Application Data
(63) Continuation of application No. 10/478,274, filed on Jul. 23, 2004, now abandoned.
(60) Provisional application No. 60/292,131, filed on May 18, 2001.

(51) Int. Cl. A61B 5/00

(52) U.S. Cl. 600/309; 600/310; 600/316

(57) ABSTRACT

The present invention relates to a system and method for monitoring or treating a health condition. The method can include accessing a biological fluid, evaluating an analyte in the fluid, presenting a measurement parameter based on the evaluation, and/or administering a treatment based on the evaluation. The measurement parameter can prompt administering a treatment. The system can include a fluid handling device, a display and/or alert device, and/or an administration device. This abstract is provided for searching purposes only and is not meant for construing the claims.

This application is being filed as a PCT International Patent application in the name of SpectRx, Inc., a U.S. national corporation (applicant for all countries except the US), and Mark Faupel, et al., U.S. residents (applicant for the US only), on 20 May 2002.

### FIELD OF THE INVENTION

The present invention relates to a system and method for monitoring or treating a health condition.

### BACKGROUND OF THE INVENTION

Many health conditions involve variations in the amount of one or more analytes in a biological fluid. These health conditions includes serious metabolic disorders. For example, diabetes involves variations in the amounts of both insulin and glucose in different body tissues. Management of diabetes typically includes determining a blood glucose level and administering a dose of insulin to avoid or correct an elevated level of glucose.

No method or system exists for monitoring analytes involved in this or numerous other health conditions. For example, ninety percent of age-associated skin problems are due to photo-damage resulting in undesirable cutaneous changes. As yet, no reliable method or system exists for monitoring analytes such as phospholipids, antioxidants, oxidizing agents, and the like that are implicated in age-related skin health conditions. Further, treatments for such skin health conditions are nearly always dispensed based on qualitative criteria that do not necessarily reflect analytic levels.

### SUMMARY OF THE INVENTION

The present invention relates to a system and method for monitoring or treating a health condition. The method can include accessing a biological fluid, evaluating an analyte in the fluid, presenting a measurement parameter based on the evaluation, and/or administering a treatment based on the evaluation. The measurement parameter can prompt administering a treatment. The system can include a fluid handling device, a display and/or alert device, and/or an administration device.

### BRIEF DESCRIPTION OF THE FIGURES

FIG. 1 is a flow chart showing several possible embodiments of the method of the present invention.

FIG. 2 is a diagrammatic representation of an embodiment of the system of the present invention including both a display and/or alert device and the optional administration device.

FIG. 3 is a diagrammatic representation of an embodiment of the system of the present invention.

### DETAILED DESCRIPTION OF THE INVENTION

The present invention includes a system and a method for evaluating and/or treating a health condition through measuring an analyte of a biological fluid. The system and method evaluate the quantity and/or quality of the analyte and, typically, couple this to administering a treatment based on the evaluation. As such, the present invention can be applied in both in vitro and/or in vivo settings. In either setting, the present invention can be used as an integral diagnostic tool as part of an ongoing evaluation of treatment. Furthermore, evaluation and administration can be on a single use basis, on a continuous use basis, or a combination of both.

Definitions

As used herein, the phrase “health condition” refers to a condition of a subject that can be treated to enhance the health, comfort, and/or well being of the subject The health condition can be a serious metabolic disorder, such as diabetes, atherosclerosis, or a dermatological disorder such as rosacae, psoriasis or seborrheic dermatitis, or eczema The health condition can also be a disorder that affects more the comfort or appearance of the subject rather then seriously affecting their life. These less serious disorders include skin disorders such as dry skin, wrinkles, acne, and the like.

As used herein, the term “treating” refers to diagnosing or determining susceptibility to a health condition; determining the presence or severity of a health condition; taking action to ameliorate, delay the onset of; prevent, or cure a health condition; or combinations thereof.

As used herein, the term “tissue” refers to an aggregate of cells of a particular kind, together with their intercellular substance, that forms a structural material. Preferred tissues include skin. Other suitable tissues include mucosal tissue and soft organs.

As used herein, the term “biological fluid” refers to blood serum, whole blood, interstitial fluid, lymph fluid, spinal fluid, plasma, cerebrospinal fluid, urine, prostatic fluid, bile, pancreatic secretions, or any combination of these fluids. Additional biological fluids include mucus, saliva, breast milk, tears, gastric secretions, and perspiration. The biological fluid can be a liquid or a gas. As used herein, the phrase “interstitial fluid” refers to the clear fluid that occupies the space between the cells in the body. The fluid may be even more specific. For example, the present method and system might use interstitial fluid from the filtrate of the capillary vessels as opposed to the “resident” interstitial fluid that may be normally found in contact with skin cells.

As used herein, the term “analyte” refers to the component that is being detected or measured in an analysis. In particular, the analyte can be any chemical or biological material or compound of which an individual might want to know the concentration or activity relating to the body. Glucose is a specific example of an analyte. It is a sugar suitable for passage through the skin, and individuals, for example those having diabetes, typically need to know their blood glucose levels.

Suitable analytes include hormones, proteins, metabolites, immune modulators (e.g., arachidonic acid metabolites, phospholipids, cytokines, and the like); markers for neutrophils, macrophages, senescent cells, mature keratinocytes, and the like; ubiquitin tagged proteins, extracellular matrix constituents (e.g., collagen, hyaluronic acids, elastin, or the like); salts or ions (e.g., sodium or potassium); oxidizing agents, antioxidants, oxidized species, flavonoids, oxygen free radicals metals; agricultural chemicals, pollutants; lipids, phospholipids, ceramides, glycerides, glycosphingolipids, alpha hydroxy acids, retinoic acids; carbohydrates, sucrose; glucose; sunscreens (e.g., para-aminobenzoic acid); caspase-14 enzyme; and the like. Other examples of analytes include, but are not limited to, such compounds as sodium, potassium, bilirubin, urea, ammonia, calcium, lead, iron, lithium, salicylates, pharmaceutical compounds, and the like. Additional analytes include albumin, alkaline phosphatase (ALP), alanine transaminase (ALT), aspartate aminotransferase (AST), bilirubin, calcium, carbon dioxide, chloride, chlorine, creatinine, glucose, lactate, lactic acid, blood urea nitrogen, uric acid, IL-1α receptor antagonist (IL-1α RA), matrix metalloprotein-1 (MMP-1), insulin-like growth factor-1 (IGF-1), interleukin-1α (IL-1α), interleukin-2 (IL-2), or a combination thereof.

Ranges may be expressed herein as from “about” or “approximately” one particular value and/or to “about” or “approximately” another particular value. When such a range is expressed, another embodiment comprises from the one particular value and/or to the other particular value. Similarly, when values are expressed as approximations, by use of the antecedent “about,” it will be understood that the particular value forms another embodiment.

Method for Enhancing Health by Evaluating an Analyte

The present method relates to treating a health condition of a subject and includes accessing a biological fluid, evaluating an analyte in the fluid, administering a treatment based on the evaluation, and/or presenting a measurement parameter based on the evaluation. The method typically accesses a biological fluid that contains an analyte related to one or more attributes of health. The analyte can be increased, decreased, or qualitatively different when the subject suffers from or is susceptible to diminished or ill health. Similarly, the analyte can be increased, decreased, or qualitatively different when the subject is in a state of good health. Depending on the quantity or quality of the analyte, the method can include presenting a measurement parameter. The measurement parameter can, for example, prompt administering a treatment. The evaluated level or quality of the analyte can also lead directly to administering a treatment.

Accessing and evaluating a biological fluid can each be conducted by any of a variety of known methods and using known apparatus. In an embodiment, accessing comprises making an opening in a tissue, for example, stratum corneum. Suitable methods and apparatus for accessing and/or evaluating are described, for example, in PCT Application No. PCT/US99/16226, filed Jul. 20, 1999, and entitled “System and Method for Fluid Management in a Continuous Fluid Collection and Sensor Device,” in PCT Application No. PCT/US99/20796, filed Sep. 10, 1999, and entitled “Attribute Compensation for Analyte Detection and/or Continuous Monitoring,” in PCT Application No. PCT/US00/09393, filed Apr. 7, 2000, and entitled “Assay Device for Measuring Characteristics of a Fluid on a Continual Basis,” and in PCT Application No. PCT/US00/16507, filed Jun. 15, 2000, and entitled “System and Method for Monitoring Glucose to Assist in Weight Management and Fitness Training.” The disclosures of each of these PCT Applications is incorporated herein by reference.

In an embodiment, evaluating an analyte can include determining an amount of an analyte. The amount of analyte can be determined as an absolute value, can be determined as a relative value, or can be an absolute value compared to a, for example, desired amount of the analyte. A relative amount of analyte can be determined compared to a desired amount of analyte or to a threshold amount of analyte. For example, a health condition can include amounts of analyte that vary around a desired level, that fall below a minimum level, or that exceed a maximum level. Evaluation can indicate the absolute or relative level of the analyte, or that the desired or threshold level is not present or has been exceeded.

For example, the method can access a biological fluid, such as interstitial fluid, and evaluate an analyte whose presence in interstitial fluid correlates with its blood level. The evaluation can determine the amount of analyte in the interstitial fluid and/or compare that amount to the desired amount. The desired amount can be based, for example, on the individual subject's medical or metabolic history or on levels observed for groups of subjects.

In an embodiment, evaluating an analyte can include determining a change in the quality of the analyte. For example, if an analyte is a mixture of compounds or forms of a compound, the distribution among the compounds or forms of the compound can change. This is referred to as a qualitative change in the analyte. This example of a qualitative change includes changes in the quantity of two or more individual compounds, that, in an embodiment, can be analytes themselves. By way of further example, a qualitative change in lipoproteins can result from a change in the amounts of low density and high density lipoproteins such that their ratio increases above a particular value.

The present method does not merely access a biological fluid and provide an evaluation of an analyte in that fluid. Rather, the method provides treatment and/or information that can be used to provide treatment based on the evaluation of the analyte.

In an embodiment, the present method includes presenting a measurement parameter based on the evaluation. The measurement parameter includes information that alerts the subject or a caregiver to the variance in the amount of the analyte. Presenting can include alerting the subject or caregiver to the measurement parameter and to a need for administering a treatment. Presenting can include displaying the measurement parameter. The measurement parameter can include, for example, an absolute amount of analyte; an amount of analyte relative to a predetermined desirable or threshold value; a signal indicating the amount of analyte is at, greater, or less than a predetermined desirable or threshold value; or a combination thereof The subject or caregiver then typically uses the presented parameter to determine a course of treatment. The course of treatment can be administered as part of the method.

In an embodiment, the present method includes administering a treatment based on the evaluation. Administering can bring the amount of analyte closer to the desired amount of analyte. If the evaluation indicates that the analyte is present in excess, the method can include administering a treatment that can decrease the amount of the analyte. Such a treatment can include a medication that lowers the amount of analyte or modifying the subject's behavior to lower the amount of analyte. For example, if the analyte is blood lipids, the treatment can include administering lipid lowering medicines, alterations in diet, or both. High levels of the analyte lactic acid or lactate can indicate that one of the subject's muscles is over exerted. In this situation, treatment can include resting the muscle or muscles.

If the evaluation indicates that the analyte is present at too low a level, the method can include administering a treatment to increase the amount of the analyte. Such a treatment can include medication that increases the amount of analytic, doses of the analyte itself, or modifying the subject's behavior to increase the amount of analyte, For example, if the analyte is vitamin D, the treatment can include doses of vitamin D, immersing the subject in sunlight, or both.

Administering can be accomplished by any of a variety of known methods for administering a treatment. Administering a therapeutic agent can include topical administration, oral administration, subcutaneous administration, a combination thereof. Preferably, for skin health administering is topical. Administering can include employing a delivery system for a treatment, agent, topical medicine, or other similar type of delivery. Administering can include causing a subject to alter behavior. For example, where the health condition is diabetes, the treatment can involve administration of insulin at the appropriate times for proper health maintenance. For the appropriate health condition, treatment can include administering topicals and/or agents, such as lotion, sunscreen, vitamins, or other agents that can deliver important and tangible results. Embodiments of such methods are described in further detail below. Other examples of possible topicals and/or agents include, without limitation, phospholipids, ceramides, liposomes, glyceride/sucrose, sunscreens, caspase-14 enzyme, antioxidants, flavonoids, AHA's, Retin-A, hyaluronic acid, glycosphingolids, green tea, caffeine, hydroquinone, kojic, tretinoin, and exfoliating acids. Moreover, the topical(s) and/or agent(s) may be administered individually or in combination depending on the health condition and/or desired treatment.

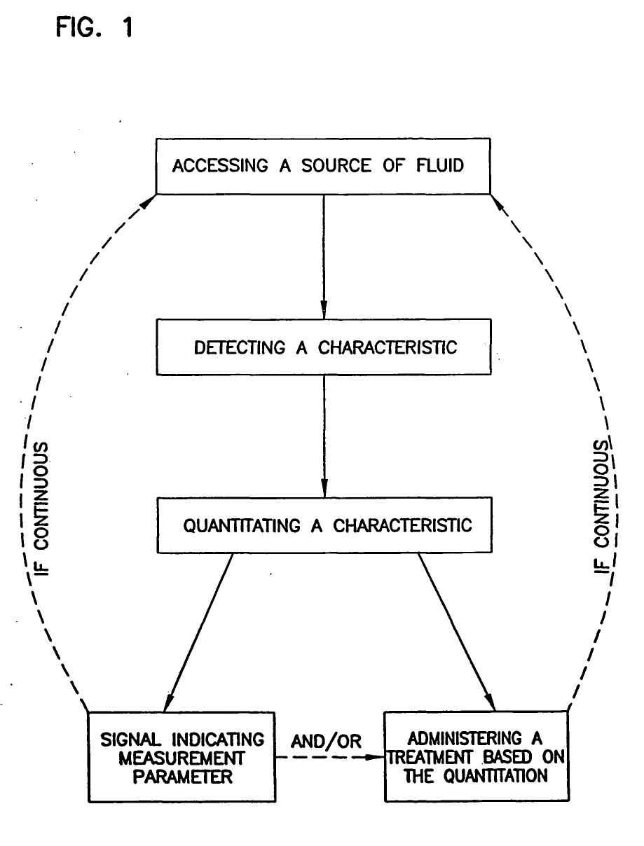

FIG. 1 schematically illustrates an embodiment of the method of the present invention. In the embodiment of FIG. 1, evaluating the analyte includes detecting and quantitating the analyte (characteristic) and presenting the measurement parameter includes a signal indicating this parameter.

This Figure illustrates that the method can operate in either discontinuous or continuous modes. In a continuous mode, presenting and/or administering are followed by additional accessing and evaluating. Such continuous operation of the method can be advantageous for health conditions in which maintaining the level of the analyte is important for health of the subject; for health conditions in which accessing, evaluating, presenting, and/or administering can readily be conducted continuously; for health conditions in which the quantity or quality of analyte can vary rapidly; or for health conditions in which variations in the quantity or quality of analyte can rapidly cause adverse health consequences for the subject.

In a discontinuous mode, presenting and/or administering conclude the method, but the method can be initiated again after a suitable interval to determine the need for additional treatment. Such discontinuous operation of the method can be advantageous for health conditions for health conditions in which one or more of accessing, evaluating, presenting, and/or administering cannot readily be conducted continuously, for health conditions in which the quantity or quality of analyte varies only slowly; or for health conditions in which variations in the quantity or quality of analyte only slowly cause adverse health consequences for the subject

Method for Enhancing Skin Health

Skin health can be affected by intrinsic and extrinsic factors, which can alter or form the quantity or quality of analyte found in fluid. Intrinsic factors include genetics, immunological function, hormones, and biological senescence. Extrinsic factors include diet, healthcare, and environmental conditions. Alterations in quantity or quality of fluid analytes due to intrinsic and extrinsic factors can be evaluated in the method of the present invention and by the system of the present invention.

Genetics can affect the quantity or quality of fluid analytes including hormones, proteins, metabolites, and the like. The quantity and quality of hormones, proteins, metabolites, and the like in biological fluid can be evaluated in the method of the present invention and by the system of the present invention.

Immunological function can affect the quantity or quality of fluid analytes including immune modulators (e.g., arachidonic acid metabolites, phospholipids, cytokines, and the like); markers for neutrophils, macrophages and the like; IL-1α RA, MMP-1, IGF-1, IL 1α, and IL2. The quantity and quality of immune modulators (e.g., arachidonic acid metabolites, phospholipids, cytokines, and the like), IL-1α RA, MMP-1, IGF-1, IL 1α, ML-2, and markers for neutrophils, macrophages and the like in biological fluid can be evaluated in the method of the present invention and by the system of the present invention.

Biological senescence can affect the quantity or quality of fluid analytes including markers for senescent cells, ubiquitin tagged proteins. The quantity and quality of markers for senescent cells, ubiquitin tagged proteins in biological fluid can be evaluated in the method of the present invention and by the system of the present invention.

Diet can affect the quantity or quality of fluid analytes including lipids, carbohydrates (e g., glucose), salts or ions (e.g., sodium or potassium). The quantity and quality of lipids, carbohydrates (e.g., glucose), and salts or ions (e.g., sodium or potassium) in biological fluid can be evaluated in the method of the present invention and by the system of the present invention.

Health care can affect the quantity or quality of fluid analytes including lipids, carbohydrates, salts or ions, medicines, and the like. The quantity and quality of lipids, carbohydrates, salts or ions, or medicines in biological fluid can be evaluated in the method of the present invention and by the system of the present invention.

Environmental conditions can affect the quantity or quality of fluid analytes including oxidizing agents, metals, agricultural chemicals, pollutants, and the like. The quantity and quality of oxidizing agents, metals, agricultural chemicals, or pollutants in biological fluid can be evaluated in the method of the present invention and by the system of the present invention.

Skin health can be affected by diseases and disorders such as rosacae, psoriasis or seborrheic dermatitis, eczema, acne, and actinic keratosis. These diseases and disorders, and their treatment, can affect the levels of fluid analytes disclosed above. The quantity and quality of fluid analytes affected by these diseases and disorders or their treatment can be evaluated in the method of the present invention and by the system of the present invention.

Particular analytes indicating skin health include IL-1α RA, MMP-1, IGF-1, IL 1α, and IL-2. The quantity and quality of these analytes can be evaluated in the method of the present invention and by the system of the present invention.

Instruments can be used pre and post treatment to measure aspects of skin health. For example, an evaporimeter such as TEWL can measure integrity of the skin's barrier function. A corneometer can measure relative skin hydration. A subumeter with lipophilic opage tape can measure casual sebum production. A ballistometer can measure skin elasticity. A chromameter can produce triplicate readings on check of skin tone. Silicone impressions can provide profilmetric analysis from readings showing more photo-damage. Also, skin biopsies could be used to quantitate changes in melanocytes and fibroblasts.

A combination of ingredients must be used to treat the broad range of problems but the skin must be monitored to determine if the combinations need to be modified as the skin improves. In situations such as these, constant monitoring might be advantageous compared to discontinuous or only a single instance of monitoring.

Embodiments of Method for Enhancing Skin Health

The present method can be applied to enhancing skin health and can include accessing a biological fluid; evaluating an analyte in the fluid; administering a treatment based on the evaluation; and/or presenting a measurement parameter based on the evaluation. For enhancing skin health, the present method typically includes accessing a biological fluid that contains an analyte indicating one or more attributes of skin health. The analyte can be increased, decreased, or qualitatively different when the skin has one or more desirable or healthy attributes or when the skin is lacking such an attribute. Depending on the level or quality of the analyte, the method can include presenting a parameter that can, for example, prompt administering a treatment, such as applying a skin care composition. The level or quality of the analyte can also or alternatively lead directly to administering a treatment, such as a skin care composition.

In an embodiment, the present method can include accessing a biological fluid, such as interstitial fluid, and evaluating an analyte in the fluid for treating a skin disease or disorder such as rosacae, psoriasis or seborrheic dermatitis, eczema, acne, or actinic keratosis. Suitable analytes include those disclosed herein. If evaluating determines a quantity or quality of the analyte indicating one or more of these skin diseases or disorders, treatment for the disease or disorder can be administered. For example, the method can include administering known therapeutic agents for these disorders. The method can include presenting a measurement parameter reflecting the quantity or quality of the analyte. This measurement parameter can be logged, recorded, or observed, and can prompt administering a treatment for the disease or disorder.

In an embodiment, the present method can include accessing a biological fluid, such as interstitial fluid, and evaluating IL-1α RA, MMP1, IGF-1, IL-1α, IL-2, or a combination thereof. If evaluating determines a quantity or quality of IL-1α RA, MMP1, IGF-1, IL-1α, IL-2, or a combination thereof indicating a need for treatment to enhance skin health, treatment to enhance skin health can be administered, For example, the method can include administering pharmaceutical agents known to affect the levels of these analytes. The method can include presenting a measurement parameter reflecting the quantity or quality of IL-1α RA, MMP1, IGF-1, IL-1α, IL,2, or a combination thereof. This measurement parameter can be logged, recorded, or observed, and can prompt administering a treatment to enhance skin health.

Table 1 presents benefits that can be accorded to skin health, processes or sources believed to be involved in achieving these benefits, and analytes or treatments that can monitor or deliver those benefits. These benefits can be achieved by the present method and system. These analytes can be evaluated in the present method and system.

TABLE 1

| | | ANALYTE OR |
|---|---|---|
| BENEFITS | PROCESS/SOURCE | TREATMENT |
| Enhanced | A) Replace “glue” to keep | A) Phospholipids; |
| Moisturization | cells together and thereby | |
| | preventing moisture loss; | |
| | B) Fill in spaces between | B) Ceramides; |
| | cells; | |
| | C) Penetrates skin and | C) Liposomes (D) |
| | releases moisture over | |
| | time (i.e., acting as a | |
| | reservoir); | |
| | D) Disperses moisture by | D) Glyceride/ |
| | increasing lipid spacing | sucrose |
| Enhanced Efficacy | Antioxidant effects | Antioxidant |
| Against Symptoms of | | |
| Sun Damage | | |
| Protection Against Sun | Guards against sun | Sunscreens |
| Damage | exposure | |
| Enhanced Barrier | Bolsters skin's barrier | Caspase-14 |
| | against the environment | enzyme |
| Strengthens Skin's | Protects from free radicals; | Antioxidants, |
| Immune Functions | prevents inflammation; | flavonoids |
| | improves circulation | |
| Exfoliation | Sloughs dead cells and | Alpha hydroxy |
| | thins stratum corneum to | acid (AHA) |
| | make skin look smoother | |
| | and firmer | |
| Wrinkle Reduction | Stimulates cell production | Retin-A |
| | to accelerate rate of cell | |
| | turnover; May also quench | |
| | oxygen free radical | |
| Firmer Skin | Acts as plasticizer or | Hyaluronic Acid |
| | lubricant for collagen to | |
| | restrict loss | |
| Thicker Skin | Potentially forms new | Glycosphingolipids |
| | elastin | |
| Reduces Redness from | | Green tea and |
| Wind, Sensitivities | | caffeine |
| Smoothes Skin and | Increases Collagen | Antioxidants, |
| Decreases Wrinkles | Production | flavonoids |
| Even Skin Tone | Regularized distribution of | Hydroquinone, |
| | melanocytes | Kojic, etc. |
| Firmer, Better | Stimulate and regulate | Combination: |
| Hydrated | fibroblast production to | retin-A and |
| | generate organized | exfoliating acids |
| | collagen, elastic and | |
| | hydrated extracellular | |
| | matrix | |
| Softer, Stronger, More | Generates keratinocyte | Combination: |
| Tolerant | maturation cycle analyte of | retin-A and |
| | healthy, younger skin | exfoliating acids |

In an embodiment, the present method can include accessing a biological fluid, such as interstitial fluid, and evaluating an analyte in the fluid for treating skin lacking adequate moisture. Suitable analytes include one or more phospholipids, one or more ceramides, and/or one or more glycerides and/or sucrose. If evaluating determines a quantity or quality of the detected analyte indicating inadequate skin moisture, treatment for inadequate skin moisture can be administered. For example, the method can include administering one or more phospholipids, one or more ceramides, one or more liposomes, one or more glycerides and/or sucrose, and/or one or more known skin moisturizers. Treatment can include alerting the subject to avoid activities that deplete the skin of moisture. The method can include presenting a measurement parameter reflecting the quantity or quality of the analyte. This measurement parameter can be logged, recorded, or observed, and can prompt administering a treatment for inadequate skin moisture.

In an embodiment, the present method can include accessing a biological fluid, such as interstitial fluid, and evaluating an analyte in the fluid for treating skin with sun damage or its symptoms. Suitable analytes include one or more antioxidants, oxidized species, or oxidizing agents. If evaluating determines a quantity or quality of the detected analyte indicating sun damage or its symptoms, treatment for sun damage or its symptoms can be administered. For example, the method can include administering one or more antioxidants. Treatment can include causing the subject to change their behavior and get out of the sun. The method can include presenting a measurement parameter reflecting the quantity or quality of the analyte. This measurement parameter can be logged, recorded, or observed, and can prompt administering a treatment for sun damage or its symptoms.

In an embodiment, the present method can include accessing a biological fluid, such as interstitial fluid, and evaluating an analyte in the fluid for preventing, delaying, or reducing the severity of sun damage or its symptoms. Suitable analytes include one or more sunscreens (e.g., para-aminobenzoic acid). If evaluating determines a quantity or quality of the detected analyte indicating inadequate protection against sun damage or its symptoms, an agent can be administered for protection against sun damage or its symptoms, such as a sunscreen. Treatment can include causing the subject to change their behavior and get out of the sun. The method can include presenting a measurement parameter reflecting the quantity or quality of the analyte. This measurement parameter can be logged, recorded, or observed, and can prompt administering a treatment for protection against sun damage or its symptoms.

In an embodiment, the present method can include accessing a biological fluid, such as interstitial fluid, and evaluating an analyte in the fluid for determining the effectiveness of skin as a barrier against the environment Suitable analytes include one or more enzymes, such as caspase-14. If evaluating determines a quantity or quality of the detected analyte indicating insufficient strength of skin as a barrier, an agent (e.g., caspase-14), can be administered for enhancing the skin's barrier properties. The method can include presenting a measurement parameter reflecting the quantity or quality of the analyte. This measurement parameter can be logged, recorded, or observed, and can prompt administering a treatment for enhancing the skin's barrier properties.

In an embodiment, the present method can include accessing a biological fluid, such as interstitial fluid, and evaluating an analyte in the fluid for determining the effectiveness of skin immune function. Suitable analytes include one or more antioxidants or flavonoids. If evaluating determines a quantity or quality of the detected analyte indicating insufficient skin immune function, an agent, such as one or more antioxidants or flavonoids, can be administered for enhancing skin immune function. The method can include presenting a measurement parameter reflecting the quantity or quality of the analyte. This measurement parameter can be logged, recorded, or observed, and can prompt administering a treatment for enhancing skin immune function.

In an embodiment, the present method can include accessing a biological fluid, such as interstitial fluid, and evaluating an analyte in the fluid for treating skin texture. Desirable skin texture features include firmness, smoothness, thickness, softness, lack of wrinkles.

That is, desirable skin is firm and taught, yet soft and smooth to the touch, is thick and wrinkle free. Undesirable skin texture features include sagging; bagging; and other forms of laxity; rough or uneven surface, such as scaly texture, coarseness (e.g. leather, stiff skin), or surface roughness (e.g. fine lines, wrinkles, skin texture); thin or translucent appearance; wrinkles including coarse wrinkles (e.g. deeper, permanent lines and furrows).

The analyte can include the level or type of one or more alpha hydroxy acids, one or more oxygen free radicals, one or more retinoic acids, one or more collagens one or more hyaluronic acids, one or more elastins, one or more glycosphingolipids, one or more antioxidants or flavonoids, one or more extracellular matrix constituents, or one or more markers for mature keratinocytes. If evaluating determines a quantity or quality of the detected analyte indicating undesirable skin texture, an agent effective for enhancing skin texture can be administered. For example, the method can include administering one or more alpha hydroxy acids, one or more retinoic acids, one or more hyaluronic acids, one or more glycosphingolipids, or one or more antioxidants or flavonoids. The method can include presenting a measurement parameter reflecting the quantity or quality of the analyte. This measurement parameter can be logged, recorded, or observed, and can prompt administering a treatment for enhancing skin texture.

Enhanced skin texture typically includes one or more of diminishment of fine lines and wrinkles; enhancement of smoothness, reduction of large pores; improvement in elasticity; improved tolerance to redness, scaling, and dryness; or normalized oil production.

In an embodiment, the present method can include accessing a biological fluid, such as interstitial fluid, and evaluating an analyte in the fluid for determining skin hue or tone. Desirable features for skin hue or tone include an even color that is without discoloration due to excess exposure to sun or wind. Undesirable features for skin hue or tone include blotching due to age spots or redness due to wind burn. Undesirable features for skin hue or tone include mottled pigmentation (e.g. melasma, freckles, age spots), sallowness, telangiectasis (i.e., red, branching skin capillaries), actinic lentigines (i.e., age spots) and actinic keratoses (i.e., pre-cancerous lesions). The analyte can include the level or type of those described herein. If evaluating determines a quantity or quality of the detected analyte indicating undesirable skin hue or tone, an agent effective for enhancing skin hue or tone can be administered. For example, the method can include administering green tea, caffeine, hydroquinone, Kojic, or a combination thereof. The method can include presenting a measurement parameter reflecting the quantity or quality of the analyte. This measurement parameter can be logged, recorded, or observed, and can prompt administering a treatment for enhancing skin hue or tone. Enhanced skin hue or tone typically includes one or more of balanced, even skin tone; reduction of age spots or hyper-pigmentation; or improved tolerance to becoming red.

Method for Enhancing Metabolic Health

The present method can be applied to enhancing metabolic health. In such an embodiment, the present method can include accessing a biological fluid; evaluating an analyte in the fluid; administering a treatment based on the evaluation; and/or presenting a measurement parameter based on the evaluations.

For enhancing metabolic health, the present method typically includes accessing a biological fluid that contains an analyte indicating one or more attributes of metabolic health. The analyte can be increased, deceased, or qualitatively different when the subject has one or more desirable or healthy metabolic states or when the subject suffers from a metabolic disease or disorder. Depending on the level or quality of the analyte, the method can include presenting a parameter that can, for example, prompt administering a treatment to enhance metabolic health. The level or quality of the analyte can also or alternatively lead directly to administering a treatment. Analytes that can reflect the presence or degree of one or more metabolic diseases or disorders include albumin, ALP, ALT, AST, bilirubin, calcium, carbon dioxide, chloride, chlorine, creatinine, glucose, lactate/lactic acid, lactate dehydrogenase (LDH), magnesium, phosphate, phosphatase, potassium, protein, sodium, triglycerides, blood urea nitrogen (BUN), and uric acid.

Embodiments of Method for Enhancing Metabolic Health

Table 2 shows the clinical significance of certain high and low analyte levels to which the present invention could be applied to monitor the health condition and treat or assist with the treatment thereof.

TABLE 2

| | | CLINICAL SIGNIFICANCE OF |
|---|---|---|
| | GENERAL CLINICAL | HIGH AND LOW ANALYTE |
| ANALYTE | SIGNIFICANCE | CONCENTRATIONS |
| Carbon | Total CO₂ measurements are | High Values (HV): |
| Dioxide | used to evaluate acid-base | Indicate respiratory acidosis with |
| | status (along with pH and | CO₂ retention or metabolic |
| | pCO₂). | alkalosis (as in prolonged |
| | Used to evaluate the total | vomiting). |
| | carbonate buffering system in | Low Values (LV): |
| | the body and acid-base status. | May indicate respiratory alkalosis |
| | | as in hyperventilation or |
| | | metabolic acidosis (as in diabetes |
| | | with ketoacidosis) |
| Chloride | Chloride is the major | High Values (HV): |
| | extracellular anion and is | Caused by dehydration, |
| | involved in maintaining water | ammonium chloride |
| | distribution, osmotic pressure | administration, renal tubule |
| | and anion-cation balance. | acidosis, and with excessive |
| | Chloride generally increases | infusion of saline. |
| | and decreases with serum or | Low Values (LV): |
| | plasma sodium. | Caused by overhydration, |
| | | congestive failure, vomiting, |
| | | chronic respiratory acidosis, |
| | | Addison's disease, metabolic |
| | | alkalosis. |
| Creatinine | Creatinine is a waste product | High Values (HV): |
| | excreted by the kidneys. | Indicate renal disease and renal |
| | Blood creatinine does not | insufficiency with decreased |
| | increase until renal function is | glomerular filtration; urinary tract |
| | greatly impaired. | obstruction; reduced renal blood |
| | Creatinine is used to evaluate | flow including congestive heart |
| | renal function and indicates | failure, shock and dehydration. |
| | glomerular filtration rate. | Low Values (LV): |
| | | May lead to small stature, |
| | | debilitation, decreased muscle |
| | | mass, and certain complex cases |
| | | of severe hepatic disease. |
| Lactate/Lactic | Lactate is the end product of | Most common cause of lactic |
| Acid | the anaerobic metabolism of | acidosis is Hypoperfusion. |
| | glucose. The concentration of | Measurement of lactic acid levels |
| | lactate in the blood is | is used to evaluate metabolic |
| | dependent on the rate of | acidosis, regional or diffuse tissue |
| | production in the muscle and | hypoperfusion, hypoxia, shock, |
| | red blood cells, and the rate of | congestive heart failure, |
| | metabolism in the liver. High | dehydration, acidosis in diabetes |
| | lactate levels result in lactic | mellitus, patients with infections, |
| | acidosis. | inflammatory states, etc. |
| | | Other causes of lactic acidosis |
| | | include: acetaminophen toxicity, |
| | | cyanide, isoniazid, propylene |
| | | glycol, phenformin, inborn errors |
| | | of metabolism, poisoning by |
| | | ethanol, methanol, salicylate, and |
| | | ethylene glycol. |
| Magnesium | Magnesium is required as a | High Values (HV): |
| | catalyst for many intracellular | Are associated with patients in |
| | enzymatic reactions | renal failure. Increased levels |
| | (particularly in carbohydrate | may be found in patients who |
| | metabolism). Increased | take magnesium salts and |
| | extracellular magnesium | magnesium-containing cathartics. |
| | depresses neural activity and | Low Values (LV): |
| | skeletal muscle contraction. | Are associated with |
| | Decreased magnesium causes | hypocalcemia, hypokalemia, |
| | irritability of the nervous | intravenous therapy, diabetes, |
| | system, peripheral | alcoholism and other types of |
| | vasodilation, and cardiac | malnutrition, malabsorption, |
| | arrhythmias. | hyperparathyroidism, dialysis, |
| | Magnesium deficiency | pregnancy, hyperaldosteronism, |
| | produces neuromuscular | and cardiac arrhythmia. |
| | disorders. It may cause | |
| | weakness, tremors, tetany, and | |
| | convulsions. | |
| Potassium | Potassium is the major cation | High Values (HV): |
| | of intracellular fluids. | [Hyperkalemia] |
| | Quantitation of potassium | Reflects inadequate renal |
| | may assist in: evaluating | excretion or inadequate |
| | electrolyte imbalance; | mobilization of potassium from |
| | following patients on diuretic | the tissues. It occurs with |
| | therapy and those with renal | hemolysis, trauma, Addison's |
| | diseases, prevent cardiac | disease, acidosis, insulin lack, |
| | arrhythmia, evaluate and treat | increased osmolality (glucose), as |
| | ketoacidosis in diabetes, etc. | well as with the administration of |
| | | potassium sparing diuretics. |
| | | Low Values (LV): |
| | | [Hypokalemia] |
| | | Potassium is decreased by |
| | | inadequate intake, excessive loss |
| | | due to diarrhea or vomiting, or |
| | | movement into the cell as in |
| | | conditions which cause alkalosis. |
| | | Found in more than 90% of |
| | | hypertensive patients with |
| | | primary aldosteronism, and |
| | | occurs with an increase in |
| | | corticosteroids. Low potassium |
| | | is much more significant with a |
| | | low pH than with a high pH. |
| Sodium | Sodium is the major cation of | High Values (HV): |
| | extracellular fluids. Sodium | [Hypernatremia] |
| | levels are regulated by the | Occurs in dehydration. |
| | kidney. | Hypernatremia without obvious |
| | Used to evaluate electrolyte, | cause may relate to Cushing's |
| | acid-base balance, water | syndrome, central or nephrogenic |
| | balance, water intoxication, | diabetes insipidus with |
| | and diagnose dehydration. | insufficient fluids, primary |
| | | aldosteronism, and other diseases. |
| | | Severe hypernatremia may be |
| | | associated with volume |
| | | contraction, lactic acidosis, |
| | | azotemia, weight loss, and |
| | | increased hematocrit as evidence |
| | | of dehydration. |
| | | Low Values (LV): |
| | | [Hyponatremia] |
| | | Occurs with nephritic syndrome, |
| | | cachexia, hypoproteinemia, |
| | | intravenous glucose infusion, and |
| | | in congestive heart failure. |
| | | Serum sodium is a predictor of |
| | | cardiovascular mortality in |
| | | patients with severe congestive |
| | | heart failure. If there is no |
| | | congestive heart failure, then |
| | | there may be hypothyroidism, |
| | | renal failure or renal sodium loss. |
| | | Sodium levels falling below 115 mmol/L |
| | | can lead to significant |
| | | neurological dysfunction with |
| | | cerebral edema and increased |
| | | intracranial pressure. |
| Triglyccrides | Represents the most abundant | High Values (HV): |
| | class of lipids (˜90%). | Evaluate hyperlipidemia, in some |
| | Nonpolar, water-insoluble | cases detect diabetes or |
| | substance which functions as | pancreatitis, hypothyroidism. |
| | an energy reservoir. Also | nephritic syndromes, and |
| | known as neutral fat or | alcoholism. Triglycerides |
| | triacylglycerol. | increase with chronic renal or |
| | | liver discase, and with obesity. |
| | | [High: >400-1000 mg/dL, |
| | | borderline: 200-400 mg/dL]. |
| Blood Urea | Urea is synthesized in the | High Values (HV): |
| Nitrogen | liver, secreted into blood, and | Occur in patients with chronic |
| (BUN) | sequestered by the kidneys for | glomerulonephritis, |
| | excretion into the urine. | pyelonephritis and other causes of |
| | BUN is used to monitor | chronic renal disease, acute renal |
| | patients on dialysis to evaluate | failure, and decreased renal |
| | renal function. | perfusion. BUN also increases |
| | | with urinary tract obstruction (as |
| | | in hyperplasia or carcinoma of |
| | | the prostrate). Increased BUN is |
| | | also caused by severe congestive |
| | | heart failure, catabolism, |
| | | tetracyclines with diuretic use, |
| | | hyperalimentation, ketoacidosis, |
| | | and dehydration as in diabetes |
| | | mellitus. [Note. Even moderate |
| | | dehydration causes BUN levels to |
| | | increase.] |
| | | Low Values (LV): |
| | | Occurs in normal pregnancy, |
| | | decreased protein intake, with |
| | | intravenous fluids, with some |
| | | antibiotics, and in some but not |
| | | all instances of liver disease. |
| Uric Acid | Uric Acid is a waste product | High Values (HV): |
| | used to diagnose gout. | Occur in renal diseases with renal |
| | | failure and prerenal azotemia as |
| | | well as gout. It is also caused by |
| | | drugs including diuretics, |
| | | pyrazinamide, ethambutol, |
| | | nicotinic acid, and aspiring in low |
| | | doses. Triglyceride increase |
| | | bears an association with |
| | | hyperuricemia, as does diabetes |
| | | mellitus and obesity, |
| | | hypertension and myocardial |
| | | infarction. (There are many other |
| | | cases which are associated with |
| | | hyperuricemia as well.) |
| | | Low Values (LV): |
| | | Caused by drugs such as high |
| | | doses of aspirin. |

In an embodiment, the present method can include accessing a biological fluid, such as interstitial fluid, and evaluating an analyte in the fluid for treating a metabolic disease such as respiratory acidosis, metabolic alkalosis, respiratory alkalosis, or metabolic acidosis. Suitable analytes include carbon dioxide. If evaluating determines a level of carbon dioxide indicating one or more of these metabolic disorder, an agent effective for treating the metabolic disorder can be administered. The method can include presenting a measurement parameter reflecting the quantity of the carbon dioxide. This measurement parameter can be logged, recorded, or observed, and can prompt administering a treatment for respiratory acidosis, metabolic alkalosis, respiratory alkalosis, or metabolic acidosis.

In an embodiment, the present method can include accessing a biological fluid, such as interstitial fluid, and evaluating an analyte in the fluid for treating metabolic disorders such as dehydration, ammonium chloride administration, renal tubule acidosis, excessive infusion of saline, overhydration, congestive failure, vomiting, chronic respiratory acidosis, Addison's disease, metabolic alkalosis; and/or disorders of water distribution, osmotic pressure, or anion-cation balance. Suitable analytes include chloride. If evaluating determines a level of chloride indicating one or more of these metabolic disorders, an agent effective for treating the disorder can be administered. Such agents include oral or intravenous administration of saline. The method can include presenting a measurement parameter reflecting the quantity of the chloride. This measurement parameter can be logged, recorded, or observed, and can prompt administering a treatment for the metabolic disorder.

In an embodiment, the present method can include accessing a biological fluid, such as interstitial fluid, and evaluating an analyte in the fluid for treating metabolic disorders such as renal disease and renal insufficiency with decreased glomerular filtration; urinary tract obstruction; reduced renal blood flow including congestive heart failure, shock and dehydration; small stature; debilitation; decreased muscle mass; or certain complex cases of severe hepatic disease. Suitable analytes include creatinine. If evaluating determines a level of creatinine indicating one or more of these metabolic disorders, an agent or treatment effective for treating the disorder can be administered. Such agents and treatments include kidney dialysis. The method can include presenting a measurement parameter reflecting the quantity of the creatinine. This measurement parameter can be logged, recorded, or observed, and can prompt administering a treatment for the metabolic disorder.

In an embodiment, the present method can include accessing a biological fluid, such as interstitial fluid, and evaluating an analyte in the fluid for treating metabolic disorders such as hypoperfusion; metabolic acidosis; regional or diffuse tissue hypoperfusion; hypoxia; shock; congestive heart failure; dehydration; acidosis in diabetes mellitus; infections or inflammatory states; or poisoning by acetaminophen, cyanide, isoniazid, propylene glycol, phenformin, ethanol, methanol, salicylate, or ethylene glycol. Suitable analytes include lactate or lactic acid. If evaluating determines a level of lactate or lactic acid indicating one or more of these metabolic disorders, an agent or treatment effective for treating the disorder can be administered. Such agents and treatments include relaxation for a muscle that has been over exerted and the like. The method can include presenting a measurement parameter reflecting the quantity of the lactate or lactic acid. This measurement parameter can be logged, recorded, or observed, and can prompt administering a treatment for the metabolic disorder.

In an embodiment, the present method can include accessing a biological fluid, such as interstitial fluid, and evaluating an analyte in the fluid for treating metabolic disorders such as depressed neural activity, depressed skeletal muscle contraction, irritability of the nervous system, peripheral vasodilation, cardiac arrhythmias, neuromuscular disorders, weakness, tremors, tetany, convulsions, renal failure, hypocalcemia, hypokalemia, intravenous therapy, diabetes, alcoholism and other types of malnutrition, malabsorption, hyperparathyroidism, dialysis, pregnancy, hyperaldosteronism, and cardiac arrhythmia. Suitable analytes include magnesium. If evaluating determines a level of magnesium indicating one or more of these metabolic disorders, an agent or treatment effective for treating the disorder can be administered. Such agents and treatments include oral or intravenous magnesium salt solutions, kidney dialysis, and the like. The method can include presenting a measurement parameter reflecting the quantity of the magnesium. This measurement parameter can be logged, recorded, or observed, and can prompt administering a treatment for the metabolic disorder.

In an embodiment, the present method can include accessing a biological fluid, such as interstitial fluid, and evaluating an analyte in the fluid for treating metabolic disorders such as electrolyte imbalance, excess diuretic therapy, renal disease, cardiac arrhythmia, ketoacidosis in diabetes, inadequate renal excretion, inadequate mobilization of potassium from the tissues, hemolysis, trauma, Addison's disease, acidosis, insulin lack, increased osmolality (e.g., of glucose), excess administration of potassium sparing diuretics, excess diarrhea or vomiting, alkalosis, hypertension with primary aldosteronism, and excess treatment with corticosteroids. Suitable analytes include potassium. If evaluating determines a level of potassium indicating one or more of these metabolic disorders, an agent or treatment effective for treating the disorder can be administered. Such agents and treatments include oral or intravenous potassium salt solutions, kidney dialysis, and the like. The method can include presenting a measurement parameter reflecting the quantity of the magnesium. This measurement parameter can be logged, recorded, or observed, and can prompt administering a treatment for the metabolic disorder.

In an embodiment, the present method can include accessing a biological fluid, such as interstitial fluid, and evaluating an analyte in the fluid for treating metabolic disorders such as imbalance of electrolyte, acid-base, or water, water intoxication; dehydration hypernatremia without obvious cause, Cushing's syndrome, central or nephrogenic diabetes insipidus with insufficient fluids, primary aldosteronism, volume contraction, lactic acidosis, azotemia, weight loss, nephritic syndrome, cachexia, hypoproteinemia, intravenous glucose infusion, congestive heart failure, hypothyroidism, renal failure or renal sodium loss, and neurological dysfunction with cerebral edema and increased intracranial pressure. Suitable analytes include sodium. If evaluating determines a level of sodium indicating one or more of these metabolic disorders, an agent or treatment effective for treating the disorder can be administered. Such agents and treatments include oral or intravenous saline, kidney dialysis, and the like. The method can include presenting a measurement parameter reflecting the quantity of the sodium. This measurement parameter can be logged, recorded, or observed, and can prompt administering a treatment for the metabolic disorder.

In an embodiment, the present method can include accessing a biological fluid, such as interstitial fluid, and evaluating an analyte in the fluid for treating metabolic disorders such as hyperlipidemia, diabetes, pancreatitis, hypothyroidism, nephritic syndromes, alcoholism, chronic renal or liver disease, and obesity. Suitable analytes include one or more triglycerides. If evaluating determines a level of or type of one or more triglycerides indicating one or more of these metabolic disorders, an agent or treatment effective for treating the disorder can be administered. The method can include presenting a measurement parameter reflecting the quantity or quality of one or more triglycerides. This measurement parameter can be logged, recorded, or observed, and can prompt administering a treatment for the metabolic disorder.

In an embodiment, the present method can include accessing a biological fluid, such as interstitial fluid, and evaluating an analyte in the fluid for monitoring patients on dialysis to determine renal function or for treating metabolic disorders such as chronic glomerulonephritis, pyelonephritis, other causes of chronic renal disease, acute renal failure, decreased renal perfusion, urinary tract obstruction (e.g., hyperplasia or carcinoma of the prostrate), congestive heart failure, catabolism, tetracyclines with diuretic use, hyperalimentation, ketoacidosis, dehydration, diabetes mellitus, normal pregnancy, decreased protein intake, with intravenous fluids, with some antibiotics, and certain instances of liver disease. Suitable analytes include blood urea nitrogen. If evaluating determines a level of blood urea nitrogen indicating one or more of these disorders, an agent or treatment effective for treating the disorder can be administered. Such agents and treatments include kidney dialysis and the like. The method can include presenting a measurement parameter reflecting the quantity of the level of blood urea nitrogen. This measurement parameter can be logged, recorded, or observed, and can prompt administering a treatment for the disorder.

In an embodiment, the present method can include accessing a biological fluid, such as interstitial fluid, and evaluating an analyte in the fluid for treating metabolic disorders such as renal diseases with renal failure and prerenal azotemia; gout; and excess treatment with diuretics, pyrazinamide, ethambutol, or nicotinic acid. Suitable analytes include uric acid. If evaluating determines a level of uric acid indicating one or more of these disorders, an agent or treatment effective for treating the metabolic disorder can be administered. Such agents and treatments include kidney dialysis and the like. The method can include presenting a measurement parameter reflecting the level of uric acid. This measurement parameter can be logged, recorded, or observed, and can prompt administering a treatment for the metabolic disorder.

System for Evaluating Analyte

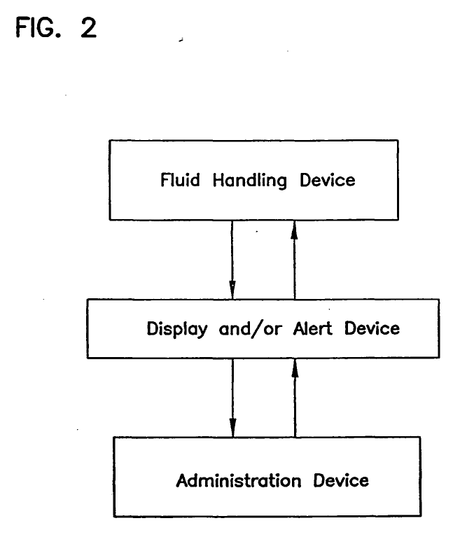

The present system for evaluating an analyte and for treating a health condition includes: a fluid handling device 1, which can obtain and evaluate the biological fluid with the analyte; a display and/or alert device 3; and/or an administration device 5. An embodiment of the present invention is illustrated in FIG. 2. The fluid handling device can be adapted and configured to obtain a biological fluid with an analyte and to evaluate the analyte. The display and alert device can be adapted and configured to display the evaluation of the analyte and alert a user to the evaluation. The administration device can be adapted and configured to administer a therapy.

Suitable fluid handling devices 1 are described, for example, in PCT Application No. PCT/US99/16226, filed Jul. 20, 1999, and entitled “System and Method for Fluid Management in a Continuous Fluid Collection and Sensor Device,” in PCT Application No. PCT/US99/20796, filed Sep. 10, 1999, and entitled “Attribute Compensation for Analyte Detection and/or Continuous Monitoring,” in PCT Application No. PCT/US00/09393, filed Apr. 7, 2000, and entitled “Assay Device for Measuring Characteristics of a Fluid on a Continual Basis,” and in PCT Application No. PCT/US00/16507, filed Jun. 15, 2000, and entitled “System and Method for Monitoring Glucose to Assist in Weight Management and Fitness Training.” The disclosures of each of these PCT Applications is incorporated herein by reference.

In an embodiment, fluid handling device 1 can include a glucose assay strip. The glucose handling strip can quantitate glucose obtained, for example, from blood or interstitial fluid. Other types of known analyte assay devices can also be employed in the present fluid handling device.

Display and/or alert device 3 can include any of a variety of known display and/or alert mechanisms. Display and alert device 3 can include a display subdevice 7 and an alert subdevice 9, which can be separate subunits. The system can include display subdevice 7, alert subdevice 9, both subdevices, or a single display and alert device 3.

The display subdevice 7 or the display and alert device 3 typically presents the absolute or relative value of the analyte in a manner that can be interpreted by the subject or caregiver. Advantageously, the display subdevice 7 or the display and alert device 3 also presents a comparison of the analyte level to the desirable or threshold levels. The display subdevice 7 or the display and/or alert device 3 can also present to the subject or caregiver a statement regarding whether the analyte level needs to be increased or decreased, whether the analyte should be administered, whether medication to alter the analyte level should be administered, or a combination thereof.

The alert subdevice 9 or the display and/or alert device 3 typically presents a detectable, attention-getting signal triggered by a predetermined variance of the analyte from a desired level, or above or below a threshold level. Any of a variety of known detectable, attention-getting signals can be employed. For example, the detectable, attention-getting signal can be a vibration, a noise, a light, and/or detectable cold or heat.

In an embodiment, the system includes an administration device 5. The administration device 5 can be coupled to the each of the other components of the system. The administration device 5 can be coupled to the liquid handling device 1, the display and/or alert device 3, each of these devices, and any subdevices 7 and 9. The administration device 5 can be triggered by the display and/or alert device to administer a treatment, if the analyte level varies by a predetermined amount from a desired level, or goes above or decreases below a threshold level. Administration can occur automatically in response to such an analyte level. Alternatively, the administration device 5 can require intervention by the subject or caregiver to activate treatment.

Any of a variety of devices for administering a therapeutic agent can be employed in the present system. For example, the administration device 5 can be a pump, an iontophoresis patch, a dispenser of topical medicine, and the like. In an embodiment, the administration device 5 includes a delivery type device such as an insulin pump or similar device that deliver topicals and/or other types of agents.

The system can be configured for continuous or discontinuous use. That is, the system can be continuously coupled to a source of biological fluid, the fluid handling device, the display and/or alert device, and/or the administration device. Each of these devices can function continuously, or as fluid becomes available, to evaluate the analyte, present a display or alert, and/or administer treatment. Alternatively, the device an be employed at selected discontinuous intervals to evaluate analyte, present a display or alert, and/or administer treatment, each portion of the system being employed only as required. Discontinuous use can include only a single use of the system.

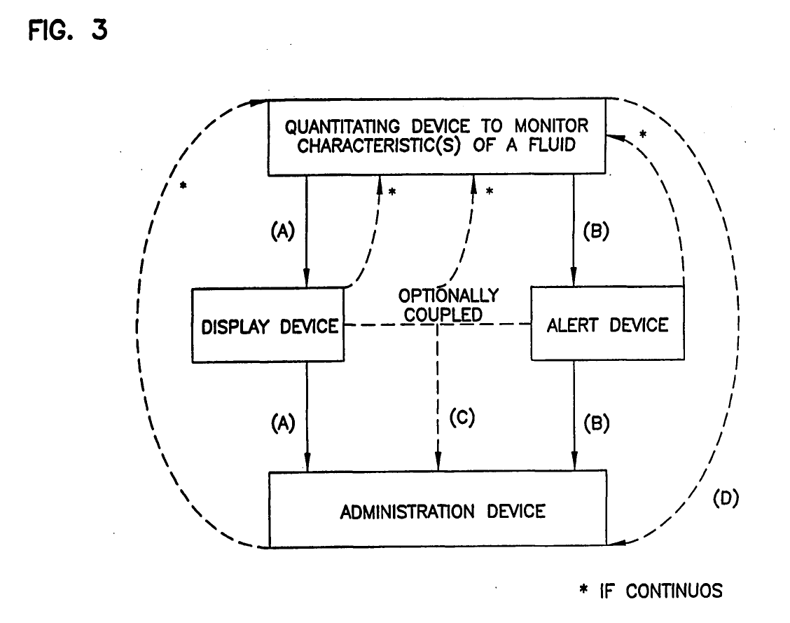

An embodiment of the system is illustrated in FIG. 3. In this embodiment, the system includes: a fluid handling device 1 in the form of a sensing device 11, which can detect and quantitate an analyte of a fluid; a display subdevice 7; an alert subdevice 9, and an administration device.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

It should also be noted that, as used in this specification and the appended claims, the phrase “adapted and configured” describes a system, apparatus, or other structure that is constructed or configured to perform a particular task or adopt a particular configuration. The phrase “adapted and configured” can be used interchangeably with other similar phrases such as arranged and configured, constructed and arranged, adapted, constructed, manufactured and arranged, and the like.

All publications and patent applications in this specification are indicating the level of ordinary skill in the art to which this invention pertains.

The invention has been described with reference to various specific and preferred embodiments and techniques. However, it should be understood that many variations and modifications may be made while remaining within the spirit and scope of the invention. The present teaching can be readily applied to other types of devices and applications that may be common to those of ordinary skill in the art Many alternatives, modifications, and variations will be apparent to those skilled in the art.

We claim:

1. A method for treating a health condition, comprising:
accessing a source of biological fluid;
evaluating an analyte of the biological fluid; and
administering a treatment based on the evaluation.

2. The method of claim 1, wherein accessing comprises making an opening in a tissue.

3. The method of claim 2, wherein the tissue comprises stratum corneum.

4. The method of claim 1, wherein the biological fluid comprises interstitial fluid, blood, serum, plasma, lymph, sweat, urine, or a combination thereof.

5. The method of claim 4, wherein the biological fluid comprises interstitial fluid.

6. The method of claim 1, wherein evaluating comprises detecting the analyte, quantitating the analyte, or a combination thereof.

7. The method of claim 6, wherein quantitating comprises determining an amount of analyte relative to a predetermined threshold value.

8. The method of claim 6, wherein quantitating comprises determining an absolute amount of analyte.

9. The method of claim 6, wherein detecting comprises determining that an amount of analyte is greater than a predetermined threshold value or is less than a predetermined threshold value.

10. The method of claim 1, wherein the treatment comprises administering a therapeutic agent or adjusting a behavior.

11. The method of claim 10, wherein administering the therapeutic agent comprises topical administration, oral administration, subcutaneous administration, or a combination thereof.

12. The method of claim 11, comprising topical administration.

13. The method of claim 1, wherein the analyte comprise albumin, ALP, ALT, AST, bilirubin, calcium, carbon dioxide, chloride, chlorine, creatinine, glucose, lactate, lactic acid, blood urea nitrogen, uric acid, IL-1α receptor antagonist, matrix metalloprotein-1, insulin-like growth factor-1, interleukin-1α, interleukin-2, or a combination thereof.

14. The method of claim 1, comprising continuous or discontinuous accessing and evaluating.

15. The method of claim 1, further comprising: presenting a measurement parameter.

16. The method of claim 15, wherein the measurement parameter comprises an amount of analyte relative to a predetermined threshold value, an absolute amount of analyte, a signal indicating an amount of analyte greater or less than a predetermined threshold value, or a combination thereof.

17. A method for treating a health condition, comprising:
accessing a source of biological fluid;
evaluating an analyte of the biological fluid; and
presenting a measurement parameter based on the evaluation.

18. The method of claim 17, wherein accessing comprises making an opening in a tissue.

19. The method of claim 18, wherein the tissue comprises stratum corneum.

20. The method of claim 17, wherein the biological fluid comprises interstitial fluid, blood, serum, plasma, lymph, sweat, urine, or a combination thereof.

21. The method of claim 20, wherein the biological fluid comprises interstitial fluid.

22. The method of claim 17, wherein evaluating comprises detecting the analyte, quantitating the analyte, or a combination thereof.

23. The method of claim 22, wherein quantitating comprises determining an amount of analyte relative to a predetermined threshold value.

24. The method of claim 22, wherein quantitating comprises determining an absolute amount of analyte.

25. The method of claim 22, wherein detecting comprises determining that an amount of analyte is greater than a predetermined threshold value or is less than a predetermined threshold value.

26. The method of claim 17, wherein the measurement parameter comprises an amount of analyte relative to a predetermined threshold value, an absolute amount of analyte, a signal indicating an amount of analyte greater or less than a predetermined threshold value, or a combination thereof.

27. The method of claim 17, wherein presenting comprises alerting a subject or caregiver to the measurement parameter and to a need for administering a treatment.

28. The method of claim 17, wherein the analyte comprise albumin, ALP, ALT, AST, bilirubin, calcium, carbon dioxide, chloride, chlorine, creatinine, glucose, lactate, lactic acid, blood urea nitrogen, uric acid, IL-1α receptor antagonist, matrix metalloprotein-1, insulin-like growth factor-1, interleukin-1α, interleukin-2, or a combination thereof

29. The method of claim 17, comprising continuous or discontinuous accessing, evaluating, presenting, or a combination thereof

30. The method of claim 17, further comprising: administering a treatment based on the evaluation.

31. The method of claim 30, wherein the treatment comprises administering a therapeutic agent or adjusting a behavior.

32. The method of claim 31, wherein administering the therapeutic agent comprises topical administration, oral administration, subcutaneous administration, or a combination thereof.

33. A system for treating a health condition, the system comprising:
a fluid handling device adapted and configured to obtain a biological fluid with an analyte and to evaluate the analyte;
a display and alert device adapted and configured to display the evaluation of the analyte and alert a user to the evaluation; and
an administration device adapted and configured to administer a therapy.

34. The system of claim 33, wherein the display and alert device comprises a display subdevice and an alert subdevice.

35. The system of claim 33, wherein the liquid handling device is adapted and configured to obtain a sample or interstitial fluid and quantitate an analyte in the interstitial fluid.
